(19) United States
(12) Patent Application Publication
(10) Pub. No.: US 2007/0260250 A1
(43) Pub. Date: Nov. 8, 2007

(54) Reinforcement of boney material surrounding a bone implant

(75) Inventors: Paul J. Wisnewski, Maple Grove, MN (US); Kimberly A. Chaffin, Woodbury, MN (US); Naim Istephanous, Roseville, MN (US); Brett R. Vegoe, Waverly, MN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc.

(21) Appl. No.: 11/429,031

(22) Filed: May 5, 2006

(51) Int. Cl. A61B 17/86

(52) U.S. Cl. 606/73; 623/23.44; 623/23.51

(57) ABSTRACT

A screw structured to engage bone has a stem with threads and an inner chamber. The inner chamber is in fluid communication with one or more ports along the stem. A polymeric material is provided with the screw. After implantation of the screw in bone, energy is applied to the polymeric material to convert the material to a more fluid form, and while in this form, the polymeric material is flowed from the inner chamber and out through the one or more ports into boney material to provide reinforcement.

### BACKGROUND

The present invention relates to a prosthetic device and manner of using the same, and more particularly, but not exclusively, relates to a technique to reinforce bone engaged by an implant.

The use of prosthetic implants to address orthopedic injuries and ailments has become commonplace. Nonetheless, there is an ever-present challenge to provide more secure and reliable implant constructs to improve procedure outcome. Thus, there is a need for additional contributions in this area of technology.

### SUMMARY

One embodiment of the present application is a unique implantation technique. Other embodiments include unique methods, systems, devices, kits, and apparatus involving an implantable prosthesis.

In one embodiment, a screw having a stem with threads and an inner chamber is implanted in bone. The inner chamber is in fluid communication with the bone through one or more port openings along the stem. A polymeric material is provided in a first state. Energy is applied to the polymeric material to convert the polymeric material to a more fluid second state. The polymeric material in the second state is flowed from the inner chamber through the one or more openings into boney material to provide reinforcement. The polymeric material returns to the first state after placement in the boney material. In one form, the second state is a softer solid phase of the polymeric material relative to a more rigid solid phase of the polymeric material in the first state and/or the second state of the polymeric material is at least partially liquid as opposed to a solid first state of the polymeric material.

In another embodiment, a screw is structured for implantation into a bone where the screw has a stem with threads and an inner chamber. The inner chamber of the screw is in fluid communication with the bone through one or more ports along the stem. A device is used to heat a polymeric material from a rigid state to a more fluid state. The polymeric material in the more fluid state is flowed from the inner chamber through the ports into the boney material to provide reinforcement.

In yet another embodiment, a screw is structured to engage bone that includes a head portion, a threaded stem opposite the head portion, and an inner chamber along the stem. The inner chamber is in fluid communication with the bone through one or more ports along the stem. A polymeric material that includes polycaprolactone is positioned within the inner chamber of the screw for delivery through the one or more ports.

In still another embodiment, a screw having a stem with threads and an inner chamber is implanted in bone. The inner chamber is in fluid communication with the bone through one or more ports along the stem. A polymeric material is provided and is heated to the make the material more fluid. The polymeric material is flowed from the inner chamber through the ports into the boney material to provide reinforcement of the screw implanted in the bone.

In a further embodiment, a polymeric material is injected into boney material through a passageway in a screw or other bone-penetrating implant. For one form, the polymeric material is a blend of one or more low molecular weight polymers and one or more high molecular weight polymers, that are more viscous than the low molecular weight polymers. The softness of the high molecular weight material is changed by heating to facilitate injection into the boney tissue, and the low molecular weight material is selected to lubricate the injection of the high molecular weight material once softened by heating.

In still a further embodiment, a material to reinforce bone includes a blend of polymers selected to depress crystallization temperature after initial melting at a relatively higher temperature to facilitate injection into boney material through an implant. Upon cooling to the depressed crystallization temperature, the material returns to a more rigid state to provide reinforcement. In one particular version of this form, the material includes a copolymer blend of polyglycolide and polycaprolactone.

One object of the present application is to provide a unique implantation technique.

Alternatively or additionally, another object of the present application is to provide a unique prosthetic method, system, device, instrument, kit, and/or apparatus.

Further embodiments, forms, features, aspects, benefits, objects, and advantages of the present application shall become apparent from the detailed description and figures provided herewith.

### BRIEF DESCRIPTION OF THE DRAWING

FIG. 1 is a partially sectional and partially diagrammatic view of a system for reinforcing an implant in bone.

FIG. 2 is a partially sectional and partially diagrammatic view of another system for reinforcing an implant in bone.

FIG. 3 is a partially sectional and partially diagrammatic view of still another system for reinforcing an implant in bone.

FIG. 4 is a graph of heat flow versus temperature.

FIG. 5 is a graph of heat flow versus time.

### DETAILED DESCRIPTION OF REPRESENTATIVE EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

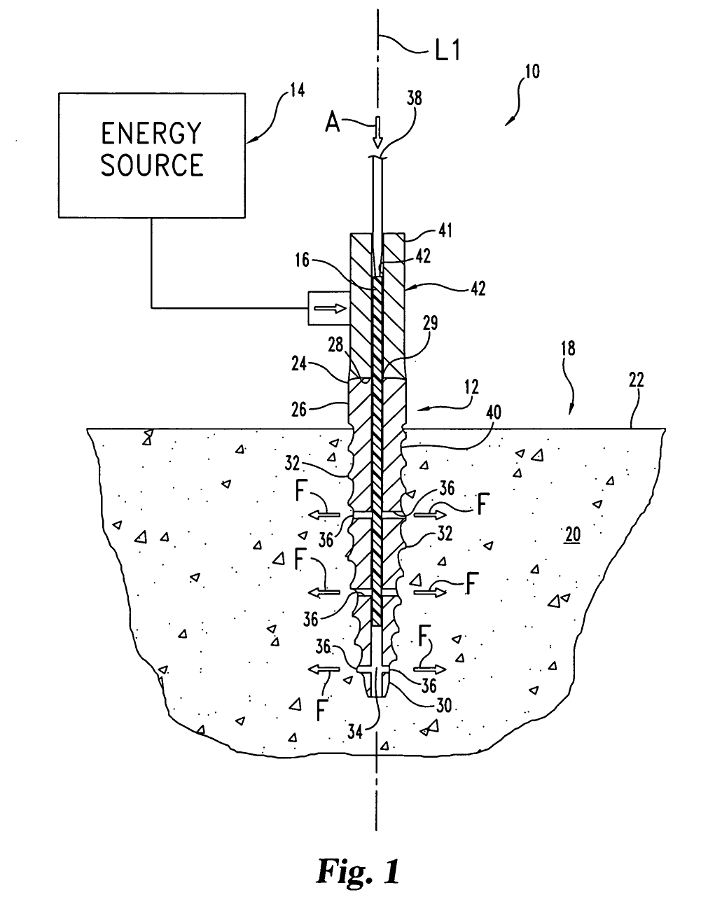

FIG. 1 depicts a reinforcement system 10 according to one embodiment of the current invention. The reinforcement system 10 comprises a screw 12, an energy source 14, and an organic polymeric material 16. Screw 12 and polymeric material 16 are shown in section, while energy source 14 is represented schematically. The reinforcement system 10 is generally utilized when the screw 12 is implanted into a bone 18. The screw 12 is made of stainless steel, titanium or alloy thereof, or such other biocompatible material as would be suitable for the intended application. It should be appreciated, that the reinforcement system 10 may be used with human bone, non-human bone, and/or other suitable hard tissue. Bone 18 may be healthy but otherwise selected for implantation, ,may be fractured, or may have some other form of disorder. In one form, bone 18 is a vertebra or sacrum of the spinal column. Screw 12 can be used alone or with one or more other components of a prosthetic construct, such as other screws, rods, bone hooks, pins, wires, plates, or the like that may or may not be mechanically interconnected. In one alternative embodiment a tissue-penetrating fastener other than a threaded screw is utilized in system 10.

The bone 18 is comprised of an outer boney material 22 and an inner boney material 20. The outer material 22 is typically dense in texture and referred to as compact tissue, and may be referred to as cortical bone particularly for a vertebral form. The inner material 20 includes of slender fibers and lamellae that join together to form a reticular structure referred to as cancellous tissue that is porous relative to compact tissue, and may be referred to as spongy bone particularly for a vertebral form. The compact tissue is located on the exterior of the bone 18 while the cancellous tissue is in the interior. The relative quantities of the tissues vary in different bones and in different areas of the same bone depending on the amount of strength required and/or other factors.

The screw 12 has a longitudinal centerline axis L, and includes a head 24 and a stem 26. The stem 26 is position opposite head 24 along axis L. The stem 26 includes outer surface 40 defining threads 32. It should also be appreciated that threads 32 may be located substantially along the entire outer surface 40, or only a portion of it, as would occur to one skilled in the art. The threads 32 engage the boney materials 20 and 22 to anchor the screw 12 within the bone 18. It should be appreciated that in other embodiments a different type of bone-penetrating fastener may be used in lieu of screw 12 that does not have threading.

The head 24 is located at a proximal end 28 of the screw 12, and stem 26 terminates at a distal end 30 of screw 12. The head 24 includes an opening 29 into an inner cannular chamber 34 defined within the stem 26, which is at least partially filled by material 16 in FIG. 1. It should be appreciated that the head 24 and/or the opening 29 may be formed to receive and/or engage an instrument to facilitate implantation of the screw 12 into the bone 18. It should also be appreciated that the head 24 and/or opening 29 may be formed to receive a delivery device that delivers reinforcement agents, steroids, medications, or any other material in addition to material 16, as may occur to one skilled in the art. More particularly, as depicted in FIG. 1, system 10 includes a heating block 41 that defines a cannular passageway 42 to receive material 16 therethrough. Passageway 42 aligns with opening 29 and chamber 34. Heating block 41 contacts proximal end 28 of screw 12 when aligned as illustrated in FIG. 1.

The inner chamber 34 is in fluid communication with several ports 36. The surface 40 defines openings for each of ports 36. The inner chamber 34 is approximately concentric with the longitudinal centerline axis L of screw 12 and extends from the opening 29 at the proximal end 28 through the distal end 30 of the screw 12. Ports 36 are in fluid communication with chamber 34, and correspondingly opening 29. When the screw 12 is implanted in the bone 18, ports 36 allow for communication of material from the inner chamber 34 of the screw 12 to the boney material 20 and/or 22 of bone 18.

The energy source 14 supplies energy to convert the polymeric material 16 to a more fluid state by raising temperature thereof. The energy source 14 provides for heating of material 16 to soften and/or melt it by applying one or more forms of energy such as radiant, conductive, or convective thermal energy; ultrasonic or other acoustic energy; radio frequency energy; or any other energy type as would occur to those skilled in the art to cause heating of material 16. For the depicted embodiment, the energy source 14 makes contact with block 41 to apply energy that results in heating of the polymeric material 16. In other embodiments, the energy source 14 may or may not be mechanically coupled to or make contact with the block 41, the screw 12 and/or any delivery/storage device containing the polymeric material 16.

The polymeric material 16 is initially loaded into the cannular passageway 42 in a solid state, and is urged into the inner chamber 34 of the screw 12 through opening 29 by applying force with a tamp 38 in the direction indicated by arrow A. Tamp 38 may be applied in this manner before, during, and/or after heating of polymeric material 16 by application of energy from the energy source 14. The tamp 38 is shaped and sized to complement passageway 42 so that it directs most, if not all of the material 16 into chamber 34. In other embodiments, some or all of the polymeric material 16 may be stored in the inner chamber 34 and/or ports 36 of the screw 12 prior to heating.

In one preferred embodiment, the polymeric material 16 includes polycaprolactone. In a more preferred embodiment, polymeric material 16 essentially consists of polycaprolactone. In an even more preferred embodiment, polymeric material 16 consists of polycaprolactone. In another more preferred embodiment, the polymeric material 16 is composed of a blend of a low-molecular weight polymer and a high molecular weight polymer. In still another even more preferred embodiment, a copolymer form of material 16 includes a low molecular weight/low viscosity polymer and the high molecular weight/high viscosity polymer comprised of polycaprolactone. While some preferred embodiments include a bioabsorbable form of polymeric material 16 (such as polycaprolactone), polymeric material 16 is not limited to bioabsorbable polymers. As an alternative or addition to tamp 38, the polymeric material 16 may be urged out of chamber 34 and ports 36 by applying a pressurized gas, a solution of saline, or such other suitable material or device.

In operation, the bone 18 is exposed and prepared during a surgical procedure, and a pilot hole is formed in the bone 18 using standard techniques. Next, the screw 12 is implanted into the bone 18 such that the threads 32 engage the boney materials 20 and 22 to treat an injury or disease. After placement of screw 12, block 41 is positioned to align passageway 42 with opening 29 and chamber 34 and the polymeric material 16 is inserted therein. The energy source 14 is activated to heat the polymeric material 16 contained within the passageway 42 to a softer or more fluid state. This change in state may be from a more rigid solid phase to a softer, more flowable sold phase, from a solid phase to a liquid phase, or a combination of these—just to name a few possibilities. The tamp 38 is used to push the polymeric material 16 out of the passageway 42 as it changes state, through the opening 29 in the head 24 of the screw 12, into the inner chamber 34 of the stem 26, and out through ports 36 as represented by arrows F. As the polymeric material 16 exits ports 36, it contacts the boney materials 20 and/or 22, occupying space in bone 18 proximate to stem 26, as facilitated by its more fluid state due to heating. After being placed in contact with the boney material, the polymeric material cools below a threshold temperature, returning to its more rigid state before heating to reinforce the bone and implant. In some preferred embodiments such as those including polycaprolactone, at least a portion of polymeric material 16 is bioabsorbed over time. In some more preferred embodiments, bioabsorption of substantially all of polymeric material 16 used to reinforce bone takes place within a time period of 6 to 12 months.

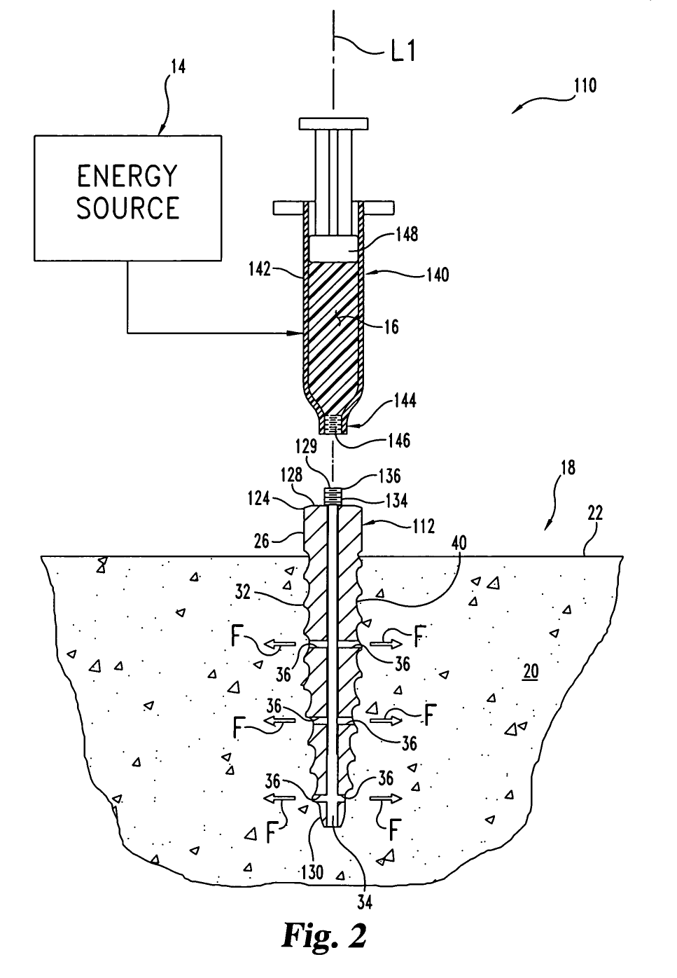

FIG. 2 depicts a reinforcement system 110 according to another embodiment of the current invention; where like reference numerals indicate like features previously described. The reinforcement system 110 comprises a screw 112, the energy source 14, and the organic polymeric material 16. Screw 112 and polymeric material 16 are shown in section, while energy source 114 is represented schematically. The screw 112 includes a head 124 and the stem 26 previously described, and may be made of material like that used to make screw 12.

The head 124 is located at a proximal end 128 of the screw 112, and the stem 26 terminates at a distal end 130 of screw 12. The head 124 includes a threaded connector 134 that defines an opening 129 into the inner chamber 34 within the stem 26. The threaded connector 134 is approximately concentric with a longitudinal centerline axis L1 of the stem 26 and is coupled to the head 124 of the screw 112. The threaded connector 134 is generally tubular in shape and includes an inlet 136 to receive the polymeric material 16 into chamber 34. It should be appreciated that the threaded connector 134 can be engaged by a device to load polymeric material 16 into chamber 34 and/or any other type of material as desired.

More particularly, as depicted in FIG. 2, system 110 includes a syringe 140 that defines a hollow body 142 partly filed with polymeric material 16. Syringe 140 has an outlet with a threaded connector 144 to engage threading of connector 134. Syringe also includes a plunger 148 to push material 16 out of body 142 into chamber 34 via opening 129. As described in connection with system 10, polymeric material 16 is heated with energy source 14 to be converted to a more flowable, less rigid state for injection through chamber 34 and ports 36. It should be appreciated that in different embodiments, the threaded connector 134 may utilize coupling methods other than threads, such as a luer lock, spring-loaded bayonet connector, or the like.

In operation, the bone 18 is exposed and screw 112 is implanted in the same manner as screw 12. After loading material 16 into body 142 of syringe 140, connector 144 is threaded on connector 134 and energy from source 14 is applied to raise the temperature of material 16 to change its state. During or after softening and/or melting, material 16 is injected from syringe 140 into chamber 34 of screw 112, and correspondingly exits chamber 34 through ports 36. The material 16 flowing through ports 36 occupies space in bone 18 surrounding screw 112 and cools, returning to its more rigid state to provide reinforcement.

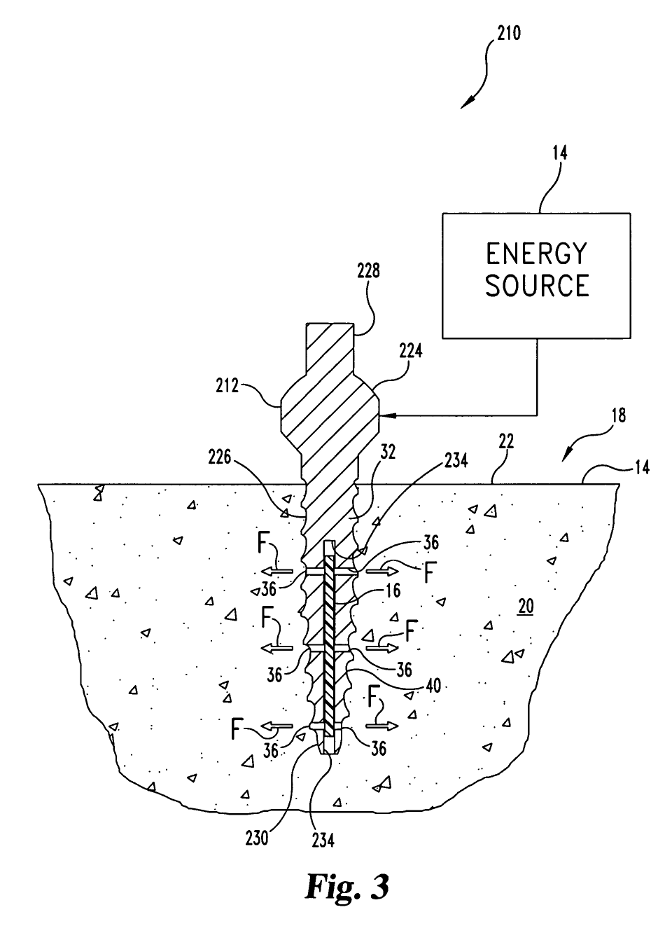

FIG. 3 depicts a reinforcement system 210 according to another embodiment of the current invention; where like reference numerals indicate like features previously described. Screw 212 and polymeric material 16 are shown in section, while energy source 214 is represented schematically. The reinforcement system 210 comprises a screw 212, energy source 14, and polymeric material 16. Screw 212 is can be made of the same material as screw 12 or screw 112. The screw 212 includes a head 224 and a stem 226 opposite head 224. The head 224 is located at a proximal end 228 of the screw 212, and stem 226 terminates at a distal end 230 of the screw 212. The head 224 may be formed to receive and/or engage an instrument to facilitate implantation of the screw 212 into the bone 18. It should also be appreciated that the head 224 may be formed to receive and/or engage an energy source 14 and installation tool.

The stem 226 includes an inner chamber 234 in fluid communication with several ports 36. Openings of ports 36 are defined by the outer surface 40 of stem 226, which also defines threads 32 as previously described. Chamber 234 extends through stem 226 to the distal end 230 of the screw 212. It should be appreciated that the inner chamber 234 may extend from within the proximal end 228 of the screw 212 to within the distal end 230 of the stem 226. The polymeric material 16 is initially positioned within the inner chamber 234 of the stem 226, being preloaded into screw 212 before implantation in bone 18.

In operation, the screw 212 is implanted into bone 18 in a standard manner, carrying polymeric material 16 in chamber 234 with it. After placement of the screw 212 in bone 18, the energy source 14 is activated to heat the polymeric material 16 contained within the inner chamber 234 of the screw 212 to a softer or more fluid state that may be a softer solid and/or liquid. In system 210, the form of energy provided by source 14 is suitable to raise the temperature of material 16 in chamber 234 through head 224, such as thermal conduction—to name just one example. As the polymeric material 16 softens and/or melts, it flows out through ports 36 as represented by arrows F. The outward flowing material 16 contacts surrounding boney materials 20 and/or 22 and occupies adjacent space within bone 18. After being placed in contact with the boney material, the polymeric material cools below a threshold temperature, returning to its initial more rigid state before heating to reinforce the bone and implant. In one preferred form of system 210, material 16 substantially changes state form a solid to a liquid during heating and returns to a solid state after cooling to provide reinforcement. However, it should be appreciated that any of the types of polymeric material 16 or equivalents thereto, as described in connection with system 10 can be used in system 110 or 210.

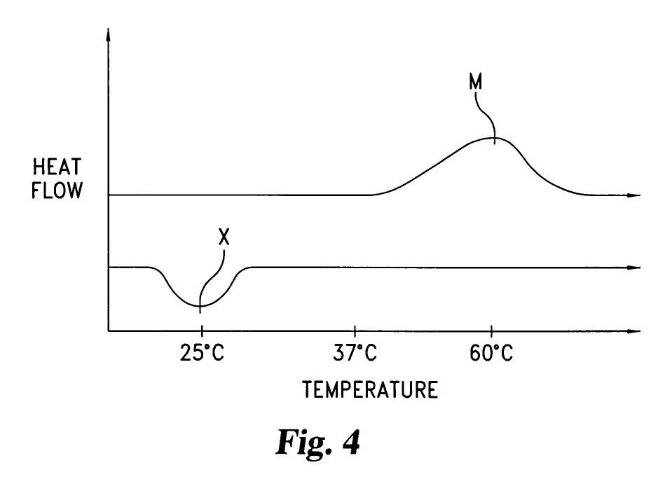

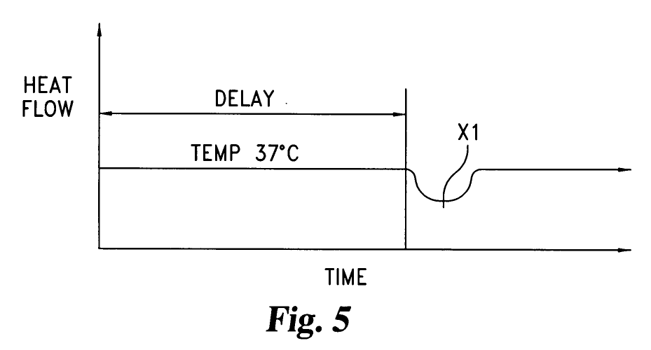

Referring to the graphs of FIGS. 4 and 5, one further preferred form of material 16 is discussed that may be desired in certain applications, and may be used to provide reinforcement in the previously described systems. For this form, material 16 includes a blend of a miscible copolymer with polycaprolactone. In one particular form, the miscible copolymer is polyglcolide. In an even more preferred blend, about 90% polyglycolide and 10% polycaprolactone by weight are provided to depress the crystallization temperature. In FIG. 4, an initial melting point of about 60 degrees Celsius resulted as indicated by the maxima M. After melting, as the blend cooled, it did not resolidify until a crystallization temperature of about 25 degrees Celsius was reached as represented by the minima X in FIG. 4. This supercooling can be provided by a variety of different blends that may or may not include Barium Sulfate for contrast. With such blends, material 16 is heated through melt and then cooled to nominal body temperature (37 degrees Celsius), while remaining a liquid. Crystallization occurs a period of time later depending on the blend. FIG. 5 illustrates this delay relative to the time of crystallization indicated at minima X1.

In another embodiment, a form of material 16 preferred for some applications is comprised of a blend of a low molecular weight/low viscosity polymer and a comparatively high molecular weight/high viscosity polymer. The lower weight polymer is selected to provide lubrication for the higher weight, more viscous polymer as it is injected into boney material from the bone implant. This alternative can also be used in previously described systems as material 16. In one more preferred form of this blend, the high molecular weight copolymer is polycaprolactone.

In still another embodiment of the current invention, a screw is implanted in a bone where the screw includes a stem, threads to engage the bone, and an inner chamber in fluid communication with one or more ports along the stem. A polymeric material in a first state is provided. Energy is applied to the polymeric material to convert the polymeric material from the first state to a second state where the second state is more fluid than the first state. The polymeric material in the second state is flowed from the inner chamber out of the one or more ports into boney material inside the bone to provide reinforcement.

In a further embodiment of the current invention, the reinforcement system comprises a screw having an inner chamber and one or more ports through an outer surface of the screw. The screw is structured for implantation into bone to place the one or more ports in fluid communication with boney material inside the bone. A means for heating a polymeric material is provided to change the polymeric material from a more rigid solid state to a more fluid state. A means for flowing the polymeric material in the more fluid state is provided to facilitate movement of the polymeric material from the inner chamber through the one or more ports to contact with boney material inside the bone. The polymeric material returns to the more rigid state to reinforce the boney material.

In yet a further embodiment of the current invention, the reinforcement system comprises a screw structured to engage human bone where the screw includes a threaded stem portion opposite a head portion and an inner chamber in fluid communication with one or more ports positioned along the threaded stem. A polymeric material is positioned within the inner chamber and is composed of polycaprolactone.

In still a further embodiment of the current invention, a screw is implanted in a bone where the screw includes a stem with at least a portion of the stem having threads to engage the bone, and an inner chamber in fluid communication with one or more ports along the stem. A polymeric material is provided and is heated to make the polymeric material more fluid. The polymeric material is flowed from the inner chamber, out of the one or more ports, and into a boney material inside the bone to provide reinforcement

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A method, comprising:
implanting a screw in a bone, the screw includes a stem, at least a portion of the stem defining threads to engage the bone, and the screw defines an inner chamber in fluid communication with one or more ports positioned along the stem;
providing a polymeric material in a first state;
applying energy to the polymeric material to convert the polymeric material from the first state to a second state, the second state being more fluid than the first state; and
flowing the polymeric material in the second state from the inner chamber out of the one or more ports into boney material inside the bone to provide reinforcement.

2. The method of claim 1, wherein the applying of the energy heats the polymeric material to transition from a solid phase to a liquid phase, the solid phase being the first state of the polymeric material and the liquid phase being the second state of the polymeric material.

3. The method of claim 2, wherein the transition form the solid phase to the liquid phase occurs at a first temperature, the polymeric material remaining in the liquid phase while cooling to a second temperature below the first temperature, the polymeric material in the liquid phase contacting the boney material while equal to or less than the second temperature and solidifying after the contacting of the boney material.

4. The method of claim 1, wherein the applying of energy converts the polymeric material from a first solid phase to a second solid phase, the first solid phase being more rigid than the second solid phase, the first solid phase being the first state of the polymeric material and the second solid phase being the second state of the polymeric material.

5. The method of claim 1, wherein the polymeric material includes polycaprolactone and the energy includes at least one of ultrasonic energy, radio frequency energy, and thermal energy to heat the polymeric material.

6. The method of claim 1, which includes performing the applying of the energy to heat the polymeric material while the polymeric material is positioned in the inner chamber.

7. The method of claim 1, wherein the screw includes a head opposite the stem and the head defines an opening into the inner chamber, and further comprising:
initiating the applying of the energy to heat the polymeric material; and
after the initiating, inserting the polymeric material into the inner chamber through the opening.

8. The method of claim 1, wherein the polymeric material includes a blend of a high molecular weight polymer and a low molecular weight polymer, the low molecular weight providing lubrication to flow the high molecular weight polymer through the one or more ports while the polymeric material is in the second form.

9. The method of claim 1, which includes:
providing the screw with the polymeric material positioned in the inner chamber in a solid state, the solid state being the first state of the polymeric material;
heating the polymeric material in the chamber by the applying of the energy to provide the polymeric material in the second state; and
after the heating, the flowing of the polymeric material in the second state including pushing the polymeric material out of the inner chamber through the one or more ports.

10. The method of claim 1, wherein the polymeric material is bioabsorbable over a time period in a range of six months through one year in the human body.

11. An apparatus, comprising:
a screw defining an inner chamber with one or more ports through an outer surface of the screw, the screw being structured for implantation into a bone to place the one or more ports in fluid communication with boney material inside the bone;
means for heating a polymeric material to change the polymeric material from a rigid solid state to a more fluid state, the fluid state being more fluid than the rigid state; and
means for flowing the polymeric material in the more fluid state from the inner chamber through the one or more ports to contact the boney material inside the bone and return to the more rigid state to reinforce the boney material.

12. An apparatus, comprising:
a screw structured to engage human bone, the screw including a threaded stem portion opposite a head portion, the screw defining an inner chamber in fluid communication with one or more ports positioned along the threaded stem; and
a polymeric material positioned in the inner chamber, the polymeric material including polycaprolactone.

13. The apparatus of claim 12, wherein the polymeric material includes a blend of the polycaprolactone and another polymer having a lower molecular weight than the polycaprolactone to provide lubrication.

14. The apparatus of claim 12, wherein the polymeric material includes a blend of the polycaprolactone and a miscible copolymer.

15. The apparatus of claim 14, wherein the copolymer is polyglycolide and the blend includes a greater amount of the polyglycolide than the polycaprolactone by weight.

16. The apparatus of claim 12, including a means for applying heat to the polymeric material.

17. A method, comprising:
implanting a screw in a bone, the screw includes a stem with at least a portion of the stem defining threads to engage the bone, the screw defines an inner chamber in fluid communication with one or more ports positioned along the stem;
providing a polymeric material;
heating the polymeric material to make the polymeric material more fluid; and
flowing the polymeric material from the inner chamber out of the one or more ports into boney material inside the bone to provide reinforcement.

18. The method of claim 17, wherein the heating converts the polymeric material from a solid phase to a liquid phase.

19. The method of claim 18, wherein the transition from the solid phase to the liquid phase occurs at a first temperature, the polymeric material remaining in the liquid phase while cooling to a second temperature below the first temperature, the polymeric material in the liquid phase contacting the boney material while equal to or less than the second temperature and solidifying after the contacting of the boney material.

20. The method of claim 17, wherein the heating converts the polymeric material from a first solid phase to a second solid phase, the second solid phase being softer than the first solid phase.

21. The method of claim 17, wherein the polymeric material includes polycaprolactone and the heating includes applying at least one of ultrasonic energy, radio frequency energy, and thermal energy.

22. The method of claim 17, which includes performing the heating while the polymeric material is positioned in the inner chamber.

23. The method of claim 17, wherein the screw includes a head, the head defines an opening into the inner chamber, and further comprising:
initiating the heating of the polymeric material; and
after the initiating, inserting the polymeric material into the inner chamber through the opening.

24. The method of claim 17, wherein the polymeric material includes a blend of a high molecular weight polymer and a low molecular weight polymer, the low molecular weight providing lubrication to flow the high molecular weight polymer through the one or more ports after the heating of the polymeric material.

25. The method of claim 17, which includes:
providing the screw with the polymeric material positioned in the inner chamber;
performing the heating of the polymeric material while in the chamber; and
after the heating, the flowing of the polymeric material including pushing the polymeric material out of the inner chamber through the one or more ports.

26. The method of claim 25, wherein the polymeric material is bioabsorbable over a time period in a range of six months through one year in the human body.
